(19) United States
(12) Patent Application Publication
(10) Pub. No.: US 2003/0111347 A1
(43) Pub. Date: Jun. 19, 2003

(54) Method for the production of colour-and/or effect-endowing multicoat paint on vehicle chassis or parts thereof

(76) Inventors: Heinrich Wonnemann, Telgte (DE); Berthold Austrup, Nordkirchen (DE); Christopher Hilger, Munster (DE); Susanne Piontek, Munster (DE)

Correspondence Address: BASF CORPORATION, ANNE GERRY SABOURIN, 26701 TELEGRAPH ROAD, SOUTHFIELD, MI, 48034-2442, US

(21) Appl. No.: 10/239,671

(22) PCT Filed: Mar. 15, 2001
(86) PCT No.: PCT/EP01/02923

(51) Int. Cl. G01L 1/20; C07K 1/26; C02F 1/469; G01F 1/64

(52) U.S. Cl. 204/506; 204/507

(57) ABSTRACT

A process for producing multicoat color and/or effect finishes on motor vehicle bodies or parts thereof (substrates), in which (I) a pigmented powder coating material is applied to the substrates, (II) the resulting powder coating film is partly or fully cured, (III) the powder coating film or powder coating is overcoated with a solid-color topcoat, with a basecoat and a clearcoat, or with a clearcoat, after which (IV) the resulting films are cured in each case individually or together with other films (wet-on-wet technique), wherein said powder coating material (A) comprises at least one carboxyl-containing polyester and (B) comprises at least one glycidyl ester of an aromatic or of a saturated or unsaturated cycloaliphatic dicarboxylic acid and/or at least one N,N,N′,N′-tetrakis(beta-hydroxyalkyl)alkanedicarboxamide, or (A) comprises at least one hydroxyl-containing polyester and (B) comprises at least one internally blocked uretdione of a diisocyanate.

The present invention relates to a novel process for producing multicoat color and/or effect finishes on motor vehicle bodies or parts thereof.

The use of powder coating materials for producing antistonechip primers and surfacer coats as part of multicoat color and/or effect finishes for the OEM finishing of motor vehicles, especially the OEM finishing of passenger automobiles, is known (cf., for example, Coatings Partner, The magazine of BASF, Powder Coatings Special, 1/2000, pages 4 to 6). In this application, the powder coating material is normally applied to a cathodically deposited and thermally cured electrodeposition coating, and baked separately. Alternatively, the powder coating material is applied to the uncured or only part-cured electrodeposition coat and is cured together with it (cf. the patent applications DE 196 06 796 A 1 or EP 0 646 420 A 1). The arrangement comprising electrodeposition coating and powder coating is also frequently referred to as the primer coat. This coat is subsequently overcoated with a color and/or effect basecoat film and a clearcoat film, which are then baked together (wet-on-wet technique). Alternatively, the primer may be provided with a solid-color topcoat film, which is then baked separately to give the solid-color topcoat.

In these modern multicoat color and/or effect finishes, the surfacers and antistonechip primers are no longer used solely for these “conventional” purposes; instead, on grounds of saving on material, attempts are made to use the surfacer coat or anti-stonechip primer to (co)establish and/or vary the depth of color and/or the shade and/or the optical effects of the basecoat or solid-color topcoat, so that in many cases the basecoat or solid-color topcoat need no longer be applied completely hidingly (cf. Römpp Lexikon Lacke und Druckfarben, Georg Thieme Verlag, Stuttgart, New York, 1998, page 124: “Hiding power”).

Corresponding pigmented powder coating materials are nowadays frequently prepared on the basis of epoxy resin/polyester. These powder coating materials are also referred to in the art as hybrid powders, since two classes of binder, in a mixture, are processed here (cf. the brochure from BASF Coatings AG entitled “Pulverlacke, Pulverlacke fër industrielle Anwendungen” [Powder coating materials, powder coatings for industrial applications], January 2000). For the utility indicated above, however, they frequently lack the necessary weathering stability, this lack being manifested disruptively by delamination and/or underchalking of the basecoat or solid-color topcoat. These problems, however, may hinder or prevent the use of the corresponding multicoat color and/or effect finishes in the particularly demanding and innovative OEM finishing of automobiles.

In the segment of the coating of utility vehicles, and especially in truck coating, from 50 to 70% of the finishes are in the achromatic color white. For the window frames, on the other hand, it is common to use the achromatic color black. For reasons of safety and presentation, trucks are commonly washed at short intervals, even daily. Many truck manufacturers therefore attempt to obtain high scratch resistance with a clearcoat, so that the finishes are not scratched by the brushes in the wash installations. For the finishing of utility vehicles, therefore, a cost-effective alternative to the customary and known multicoat color and/or effect finishes is a coating system comprising a color and/or effect powder coat—for example, a white and/or black powder coat—and a clearcoat.

The problems depicted above and associated with the inadequate weathering stability of the hybrid powder coatings are manifested here to an increased extent. This coating system can therefore be used, even as a model system for determining the suitability of powder coating materials for the production of the above-described multicoat finish comprising a basecoat or a topcoat.

The required weathering stability could be established using powder coating materials based on polyacrylates, since these give highly weathering-resistant coatings. Coating systems of this kind are known, for example, from U.S. Pat. No. 4,268,542 A 1. For instance, Example 3 of the patent describes the production of a multicoat finish by application of a black powder coating material to aluminum, application of a powder slurry clearcoat to the resulting powder coating film, and conjoint baking of the two coats. The resulting multicoat finish exhibits good leveling. Although the patent proposes further powder coating materials for use in this process, nothing is said about the weathering stability of the resulting multicoat finishes. Instead, the patent is directed essentially to a process in which for purposes of improved leveling of the multicoat finish in question pigmented powder coating films and pigmented powder slurry films are applied over one another and baked together.

In principle, transparent powder coating materials based on polyacrylates cannot be prepared alongside the customary polyester-based standard powder coating materials in one and the same plant, since this would immediately result in contamination of production.

A certain improvement in the weathering stability of coatings based on standard powder coating materials might be achievable through the use of triglycidyl isocyanurate (TGIC) as crosslinking agent. However, this compound is very toxic, and for that reason its use is increasingly being avoided.

It is an object of the present invention to find a new process for producing multicoat color and/or effect finishes on motor vehicle bodies or parts thereof, in which pulverulent coating materials are applied to the motor vehicle bodies and/or motor vehicle body parts and the resulting powder coats are cured, which process no longer has the disadvantages of the prior art but rather can be carried out using powder coating materials whose preparation does not cause any contamination of production, said process providing multicoat color and/or effect finishes which exhibit good intercoat adhesion and a high level of substrate adhesion and also particularly high weathering stability even without the use of TGIC.

Accordingly we have found the novel process for producing multicoat color and/or effect finishes on motor vehicle bodies or parts thereof (substrates), in which

(I) a pigmented powder coating material is applied to the substrates,

(II) the resulting powder coating film is partly or fully cured,

(III) the powder coating film or powder coating is overcoated with a solid-color topcoat, with a basecoat and a clearcoat, or with a clearcoat, after which

(IV) the resulting films are cured in each case individually or together with other films (wet-on-wet technique),

wherein said powder coating material

(A) comprises at least one carboxyl-containing polyester as binder and

(B) comprises at least one glycidyl ester of an aromatic or of a saturated or unsaturated cycloaliphatic dicarboxylic acid and/or at least one N,N,N′,N′-tetrakis(beta-hydroxyalkyl)alkanedicarboxamide as crosslinking agent, or

(A) comprises at least one hydroxyl-containing polyester as binder and

(B) comprises at least one internally blocked uretdione of a diisocyanate as crosslinking agent.

The novel process for producing multicoat color and/or effect finishes on motor vehicle bodies or parts thereof is referred to below as “process of the invention”.

Further subject matter of the invention will emerge from the description.

The process of the invention is used to produce multicoat color and/or effect finishes on motor vehicle bodies and parts thereof. For the purposes of the present invention, motor vehicle bodies are understood to be bodies of automobiles or of utility vehicles such as trucks or buses. Parts of motor vehicle bodies are understood in particular as attachments such as doors, hoods, wind deflectors, spoilers, wings or sills made of metal or plastic. They are referred to collectively below as substrates.

The substrates may consist of metals as commonly used in motor vehicle production, such as iron, steel, galvanized steel, or aluminum.

In the case of steel, it may be provided with primers, which are conventionally produced from electrodeposition paints (EDP). Suitable for this purpose are both anodic (AED) and cathodic (CED) electrodeposition paints, but especially CED (cf., for example, German Patent Application DE 196 06 706 A 1). In this case the substrate may also be subjected to a surface treatment: for example, galvanizing or phosphating.

In the process of the invention, the EDP coat may be cured fully before the other coats are applied. Alternatively, it may be merely dried, after which at least one further coat of the multicoat finish produced in accordance with the invention is baked together with the EDP coat (cf. the patent applications DE 196 06 706 A 1 or EP 0 646 420 A 1). Preferably, the EDP coating has a coat thickness of from 5.0 to 35, in particular from 10 to 25 μm.

In the case of aluminum, there is no need to apply an EDP primer. Instead, the surface of the aluminum may be anodically oxidized so as to form a corrosion-stable coat of alumina, a process also known to those in the art as eloxing.

The process of the invention can also be used to coat primed or unprimed plastics that are dimensionally stable at the baking temperatures employed, such as, for example, polyphenylene ethers, SMC, BMC, polyether sulfone or polyether ketone. The plastics may of course also be polymer blends, modified plastics, or fiber-reinforced plastics. In the case of unfunctionalized and/or apolar substrate surfaces, these plastics may be subjected to pretreatment in a known manner prior to coating, such as with a plasma or by flaming, or may be provided with an aqueous primer.

In the first step of the process of the invention, a color and/or effect powder coating material is applied to the substrates described above. This is done using the conventional processes and apparatus, as described, for example, in Coatings Partner, The magazine of BASF, Powder Coatings Special, 1/2000, or in the BASF Coatings AG company brochure “Pulverlacke, Pulverlacke für industrielle Anwendungen” [Powder coating materials, powder coatings for industrial applications], January 2000.

It is essential in this context that the powder coating material for use in accordance with the invention comprises either at least one carboxyl-containing polyester or at least one hydroxyl-containing polyester as binder (A).

Examples of suitable carboxyl-containing polyesters (A) for use in accordance with the invention are described in German Patent Application DE 196 06 706 A 1, column 6, line 29 to column 7, line 14, in detail. In addition to the commercial polyesters listed there, mention may also be made of Uralac® P846, P2450, P3475, P3480 or P3485 from DSM or Alftalat® AN 783 from the company Solutia.

The powder coating material based on carboxyl-containing polyesters (A), for use in accordance with the invention, comprises at least one glycidyl ester of an aromatic or of a saturated or unsaturated cycloaliphatic dicarboxylic acid and/or at least one N,N,N′,N′-tetrakis(beta-hydroxyalkyl)alkane-dicarboxamide as crosslinking agent (B). The powder coating material preferably contains at least one glycidyl ester of an aromatic or of a saturated or unsaturated cycloaliphatic dicarboxylic acid or at least one N,N,N′,N′-tetrakis(beta-hydroxyalkyl)alkane-dicarboxamide.

Examples of suitable aromatic dicarboxylic acids are phthalic acid, terephthalic acid or isophthalic acid, especially terephthalic acid and/or isophthalic acid.

Examples of suitable unsaturated cycloaliphatic dicarboxylic acids are tetrahydrophthalic acid, tetrahydroterephthalic acid or tetrahydroisophthalic acid.

Examples of suitable saturated cycloaliphatic dicarboxylic acids are hexahydrophthalic acid, hexahydroterephthalic acid or hexahydroisophthalic acid.

Of these, the aromatic dicarboxylic acids are particularly advantageous and therefore used with preference.

Examples of suitable beta-hydroxyalkyl groups are 2-hydroxyethyl, 2-hydroxypropyl or 2-hydroxy-n-butyl, of which 2-hydroxyethyl is particularly advantageous and is therefore used with preference in accordance with the invention.

Examples of suitable alkanedicarboxamides are the diamides of C₅ to C₁₆ alkanedicarboxylic acids such as succinic acid, adipic acid, sebacic acid or suberic acid, especially adipic acid.

In accordance with the invention, therefore, the diglycidyl esters of terephthalic acid and/or isophthalic acid, or N,N,N′,N′-tetrakis(beta-hydroxy-ethyl)adipamide, are used with particular preference as crosslinking agent (B).

Examples of suitable hydroxyl-containing polyesters (A) for use in accordance with the invention are polyesters prepared from the starting compounds described in detail in German Patent Application DE 196 06 706 A 1, column 6, line 29 to column 7, line 14 but choosing the proportions of polyols to polycarboxylic acids and/or polycarboxylic acid derivatives such that the resultant hydroxyl-containing polyesters (A) have an acid number of less than 10, preferably less than 5 and, in particular, less than 3 mg KOH/g. Their hydroxyl number is preferably from 20 to 250, more preferably from 25 to 200, with particular preference from 30 to 180, with very particular preference from 35 to 170 and, in particular, from 40 to 160 mg KOH/g. Hydroxyl-containing polyesters are commercial products and are sold, for example, under the brandname Uralac®, e.g., Uralac® 1480, by the company DSM.

Suitable diisocyanates for preparing the internally blocked uretdiones which constitute the crosslinking agents (B) for the hydroxyl-containing polyesters (A) are aliphatic, cycloaliphatic or aliphatic-cycloaliphatic diisocyanates. Of these, preference is given to those whose two isocyanate groups differ in reactivity. Of these, particular preference is given in turn to those whose one isocyanate group is attached to an alkyl radical and whose other isocyanate group is attached to a cycloalkyl radical.

Examples of suitable diisocyanates are isophorone diisocyanate (=5-isocyanato-1-isocyanato-methyl-1,3,3-trimethylcyclohexane), 5-isocyanato-1-(2-isocyanatoeth-1-yl)-1,3,3-trimethylcyclohexane, 5-iso-cyanato-1-(3-isocyanatoprop-1-yl)-1,3,3-trimethylcyclohexane, 5-isocyanato-(4-isocyanatobut-1-yl)-1,3,3-trimethylcyclohexane, 1-isocyanato-2-(3-isocyanatoprop-1-yl)cyclohexane, 1-isocyanato-2-(3-isocyanatoeth-1-yl)-cyclohexane, 1-isocyanato-2-(4-isocyanatobut-1-yl)-cyclohexane, 1,2-diisocyanatocyclobutane, 1,3-diisocyanatocyclobutane, 1,2-diisocyanatocyclopentane, 1,3-diisocyanatocyclopentane, 1,2-diisocyanatocyclohexane, 1,3-diisocyanatocyclohexane, 1,4-diisocyanatocyclohexane, dicyclohexylmethane 2,4′-diisocyanate, trimethylene diisocyanate, tetramethylene diisocyanate, pentamethylene diisocyanate, hexamethylene diisocyanate, ethylethylene diisocyanate, trimethylhexane diisocyanate, heptamethylene diisocyanate, or diisocyanates derived from dimeric fatty acids, as sold under the commercial designation DDI 1410 by the company Henkel and described in the patents WO 97/49745 and WO 97/49747, especially 2-heptyl-3,4-bis(9-iso-cyanatononyl)-1-pentylcyclohexane, or 1,2-, 1,4- or 1,3-bis(isocyanatomethyl)cyclohexane, 1,2-, 1,4- or 1,3-bis(2-isocyanatoeth-1-yl)cyclohexane, 1,3-bis(3-isocyanatoprop-1-yl)cyclohexane, 1,2-, 1,4- or 1,3-bis(4-isocyanatobut-1-yl)cyclohexane, or liquid bis(4-isocyanatocyclohexyl)methane with a trans/trans content of up to 30% by weight, preferably 25% by weight and in particular 20% by weight, as is described in the patents DE-A-44 14 032, GB-A-1220717, DE-A-16 18 795 or DE-A-17 93 785, preferably isophorone diisocyanate, 5-isocyanato-1-(2-isocyanatoeth-1-yl)-1,3,3-trimethylcyclohexane, 5-isocyanato-1-(3-isocyanatoprop-1-yl)-1,3,3-trimethylcyclohexane, 5-isocyanato-(4-isocyanato-but-1-yl)-1,3,3-trimethylcyclohexane, 1-isocyanato-2-(3-isocyanatoprop-1-yl)cyclohexane, 1-isocyanato-2-(3-isocyanatoeth-1-yl)cyclohexane, 1-isocyanato-2-(4-isocyanatobut-1-yl)cyclohexane, especially isophorone diisocyanate.

The preparation of the internally blocked uretdiones from the diisocyanates presents no special features as to its method, but instead takes place, for example, as described in European Patent Application EP 0 045 998 A 1.

Any free isocyanate groups still present in the internally blocked uretdiones may be blocked using appropriate blocking agents. Alternatively, these blocking agents may already be present when the internally blocked uretdiones are being prepared.

Examples of appropriate blocking agents are the blocking agents known from U.S. Pat. No. 4,444,954:

i) phenols such as phenol, cresol, xylenol, nitrophenol, chlorophenol, ethylphenol, t-butylphenol, hydroxybenzoic acid, esters of these acids, or 2,5-di-tert-butyl-4-hydroxytoluene;

ii) lactams, such as ε-caprolactam, δ-valerolactam, γ-butyrolactam or β-propiolactam;

iii) active methylenic compounds, such as diethyl malonate, dimethyl malonate, ethyl acetoacetate, methyl acetoacetate, or acetylacetone;

iv) alcohols such as methanol, ethanol, n-propanol, isopropanol, n-butanol, isobutanol, t-butanol, n-amyl alcohol, t-amyl alcohol, lauryl alcohol, ethylene glycol monomethyl ether, ethylene glycol monoethyl ether, ethylene glycol monopropyl ether, ethylene glycol monobutyl ether, diethylene glycol monomethyl ether, diethylene glycol monoethyl ether, propylene glycol monomethyl ether, methoxymethanol, glycolic acid, glycolic esters, lactic acid, lactic esters, methylolurea, methylolmelamine, diacetone alcohol, ethylene-chlorohydrin, ethylenebromohydrin, 1,3-dichloro-2-propanol, 1,4-cyclohexyldimethanol or acetocyanohydrin or diols such as ethylene glycol, diethylene glycol, propylene glycol, butylene glycol, alone or in a mixture with at least one of the alcohols;

v) mercaptans such as butyl mercaptan, hexyl mercaptan, t-butyl mercaptan, t-dodecylmercaptan, 2-mercaptobenzothiazole, thiophenol, methylthiophenol or ethylthiophenol;

vi) acid amides such as acetoanilide, aceto-anisidinamide, acrylamide, methacrylamide, acetamide, stearamide or benzamide;

vii) imides such as succinimide, phthalimide or maleimide;

viii) amines such as diphenylamine, phenylnaphthylamine, xylidine, N-phenylxylidine, carbazole, aniline, naphthylamine, butylamine, dibutylamine or butylphenylamine;

ix) imidazoles such as imidazole or 2-ethylimidazole;

x) ureas such as urea, thiourea, ethyleneurea, ethylenethiourea or 1,3-diphenylurea;

xi) carbamates such as phenyl N-phenylcarbamate or 2-oxazolidone;

xii) imines such as ethyleneimine;

xiii) oximes or ketoximes such as acetone oxime, formaldoxime, acetaldoxime, acetoxime, methyl ethyl ketoxime, diisobutyl ketoxime, diacetyl monoxime, benzophenone oxime or chlorohexanone oximes;

xiv) salts of sulfurous acid such as sodium bisulfite or potassium bisulfite;

xv) hydroxamic esters such as benzyl methacrylohydroxamate (BMH) or allyl methacrylohydroxamate; or

xvi) substituted pyrazoles, especially dimethylpyrazole, imidazoles or triazoles; and also

xvii) mixtures of these blocking agents, such as dimethylpyrazole and triazoles, malonic esters and acetoacetic esters, or dimethylpyrazole and succinimide.

Of these, the diols or the mixtures of alcohols and diols (iv) afford particular advantages and are therefore used with particular preference in accordance with the invention.

Examples of especially suitable internally blocked uretdiones of diisocyanates are known from the article by A. Wennig, J. -V. Weiβ and W. Grenda, “Polyisocyanates today and tomorrow”, European Coatings Journal (ECJ), No. 4, 1998, pages 244 to 249. They are commercially customary products, which are sold for example under the brandname Vestagon®, e.g., Vestagon® BF 1540, BF 1300 or BF 1310, by the company Hüls.

The amount of the binders (A) in the powder coating materials for use in accordance with the invention may vary very widely. It is preferably from 20 to 95, more preferably from 25 to 85, with particular preference from 30 to 75, with very particular preference from 35 to 65, and in particular from 40 to 60% by weight, based in each case on the powder coating material.

In the powder coating materials for use in accordance with the invention the crosslinking agents (B) are present preferably in an amount of from 0.5 to 20, more preferably from 1.0 to 18, with particular preference from 2.0 to 17, with very particular preference from 3.0 to 16, and in particular from 5.0 to 15% by weight, based in each case on the powder coating material.

Furthermore, the powder coating materials for use in accordance with the invention may comprise minor amounts of the conventional low molecular mass crosslinking agents containing epoxide groups, as described for example in the patent application DE 196 06 706 A 1, column 7, lines 15 to 25. The term “minor amounts” means that the curing properties and the profile of properties of the resultant powder coatings continue to be determined essentially by the crosslinking agents (B) for use in accordance with the invention.

The powder coating material for use in accordance with the invention further comprises at least one color and/or effect pigment (C).

The pigments (C) may comprise organic or inorganic compounds. On the basis of this large number of suitable pigments (C), therefore, the powder coating material for use in accordance with the invention ensures a universal breadth of application and makes it possible to realize a large number of color shades and optical effects.

As effect pigments (C) it is possible to use metal flake pigments such as commercial aluminum bronzes, aluminum bronzes chromated in accordance with DE-A-36 36 183, commercial stainless steel bronzes, and nonmetallic effect pigments, such as pearlescent and interference pigments, for example. For further details, reference is made to Römpp Lexikon Lacke und Druckfarben, Georg Thieme Verlag, 1998, pages 176 “Effect pigments” and pages 380 and 381 “Metal oxide mica pigments” to “Metal pigments”.

Examples of suitable inorganic color pigments (C) are titanium dioxide, iron oxides, and carbon black. Examples of suitable organic color pigments are thioindigo pigments, indanthrene blue, Irgalith blue, Cromophthal Red, Irgazine orange, Sicotrans yellow, Sicotan yellow, Hostaperm yellow, Paliotan yellow and Heliogen green. For further details, reference is made to Römpp Lexikon Lacke und Druckfarben, Georg Thieme Verlag, 1998, pages 180 and 181, “Iron blue pigments” to “Black iron oxide”, pages 451 to 453 “Pigments” to “Pigment volume concentration”, page 563 “Thioindigo pigments”, and page 567 “Titanium dioxide pigments”.

The fraction of the pigments (C) in the powder coating materials for use in accordance with the invention may vary very widely and is guided by the requirements of the specific case, in particular by the optical effect to be established and/or the hiding power of the particular pigments (C) used.

The powder coating material used in accordance with the invention may further comprise organic and inorganic fillers (D).

Examples of suitable organic and inorganic fillers (D) are chalk, calcium sulfates, barium sulfate, silicates such as talc or kaolin, silicas, oxides such as aluminum hydroxide or magnesium hydroxide, or organic fillers such as polyamide particles. For further details, reference is made to Römpp Lexikon Lacke und Druckfarben, Georg Thieme Verlag, 1998, pages 250 ff., “Fillers”. Further examples of suitable fillers (D) are known from German Patent Application DE 196 06 706 A 1, column 8, lines 30 to 64. They are preferably used in the amounts specified therein.

The amount of pigments (C) and, if present, of fillers (D) is preferably from 0.1 to 70, more preferably from 0.5 to 65, with particular preference from 1.0 to 60, with very particular preference from 2.0 to 55, and in particular from 2.5 to 50% by weight, based in each case on the powder coating material.

Furthermore, the powder coating material for use in accordance with the invention may additionally comprise at least one additive (E). Examples of suitable additives (E) are other oligomeric and polymeric binders different from the polyesters (A), UV absorbers, light stabilizers, free-radical scavengers, devolatilizers, slip additives, adhesion promoters, leveling agents, flow aids, corrosion inhibitors, waxes, and flatting agents. Further examples of these and other additives (E) are described in detail in the textbook “Lackadditive” by Johan Bieleman, Wiley-VCH, Weinheim, New York, 1998.

The preparation of the powder coating materials for use in accordance with the invention has no special features as to its method but instead takes place with the aid of the conventional methods and apparatus, as described, for example, in the product information bulletin from BASF Lacke+Farben AG, “Pulverlacke” [Powder coatings], 1990 or in the BASF Coatings AG brochure “Pulverlacke, Pulverlacke fur industrielle Anwendungen” [Powder coating materials, powder coatings for industrial application], January 2000.

The particle size distribution of the powder coating materials for use in accordance with the invention may vary comparatively widely and is guided by the particular intended use. Preferably, the particle size distribution is comparatively narrow, with a very low fraction of coarse particles (particle sizes above 95 μm) and of ultrafine particles (particle sizes below 5.0 μm). Particular preference is given to the use of powder coating materials having the particle size distribution described in European Patent Application EP 0 666 779 A 1.

The application of the powder coating materials also takes place in accordance with the conventional methods, as described in the two abovementioned company brochures, for example.

Following application, the powder coating films are melted and thermally cured to give the powder coatings. The curing of the powder coating films, as well, has no special features as to its method but instead takes place in accordance with the conventional thermal methods such as heating in a circulating-air oven or irradiation with IR lamps or with lamps which emit near infrared (NIR) radiation, at temperatures from 120 to 220, preferably from 130 to 220, more preferably from 150 to 200 and, in particular, from 160 to 190° C. These methods and apparatus may of course also be used for the thermal curing of the other coats which build up the multicoat finishes.

Where the powder coating material in the process of the invention has been applied to an uncured or only part-cured electrodeposition coat (EDC), at this stage of the process of the invention this coat is cured together with the powder coating film (cf. patent applications DE 196 06 706 A 1 or EP 0 646 420 A 1).

In another variant of the process of the invention, the powder coating film, applied if appropriate to an uncured or only part-cured, or to a fully cured, EDC, is melted and is not cured at all, or is only part-cured, or is not subjected to any further aftertreatment, but instead is first coated with at least one further coat, after which it is cured together with the EDC (if present) and with the further coat or coats. This is also referred to by those in the art as the “dry-on-wet” technique.

In a first preferred variant in accordance with the process of the invention, a conventional solid-color topcoat is applied to the powder coating film or powder coating. The resulting solid-color topcoat can then be cured, alone or together with the powder coating film and with the EDC if present.

In a second preferred variant of the process of the invention, a basecoat, especially an aqueous basecoat, and a clearcoat are applied to the powder coating film or powder coating. Thereafter, the resulting basecoat film and the resulting clearcoat film may be cured individually in succession. Alternatively, the basecoat film and the powder coating film may be cured together, after which the clearcoat film is cured on its own. Furthermore, the powder coating film, the basecoat film and the clearcoat film may be cured together. Preferably, the basecoat film and the clearcoat film are cured together (“classic” wet-on-wet technique).

With this variant of the process of the invention, it is possible to use essentially pure color powder coating materials and essentially pure effect basecoat materials. This spatial separation of the color and effect functions makes it possible to produce new kinds of attractive optical effects in a simple and elegant manner. The breadth of application of the process of the invention is increased still further in this case by graduating the spatial separation of the functions to a greater or lesser extent; for example, with the powder coating having a more pronounced effect-imparting effect and/or the basecoat having a more pronounced color-imparting effect.

In a third preferred variant of the process of the invention, a clearcoat is applied to the powder coating or powder coating film, after which the resulting clearcoat film is cured either together with the powder coating film or on its own.

The clearcoat film or clearcoat may also be overcoated with a high scratch resistance sealer, which may then be cured together with the clearcoat film or on its own.

In the process of the invention, the curing of the solid-color topcoat film or of the basecoat film, of the clearcoat film and/or of the sealer, but in particular of the clearcoat film, may be supplemented by actinic radiation, something which is also referred to by those skilled in the art as dual cure. In the case of the clearcoat film and/or of the sealer, curing may also take place with actinic radiation alone. In that case, the underlying coats are preferably already fully cured. For the purposes of the present invention, actinic radiation is electromagnetic radiation, such as near infrared (NIR), visible light, UV radiation or X-rays, especially UV radiation, or corpuscular radiation, such as electron beam. Suitable apparatus and processes for curing with actinic radiation are described in the patents listed below relating to clearcoats curable with actinic radiation.

Examples of suitable solid-color topcoats and basecoats, especially aqueous basecoats, which may be processed in the process of the invention are known from the patents EP 0 089 497 A 1, EP 0 256 540 A 1, EP 0 260 447 A 1, EP 0 297 576 A 1, WO 96/12747, EP 0 523 610 A 1, EP 0 228 003 A 1, EP 0 397 806 A 1, EP 0 574 417 A 1, EP 0 531 510 A 1, EP 0 581 211 A 1, EP 0 708 788 A 1, EP 0 593 454 A 1, DE-A-43 28 092 A 1, EP 0 299 148 A 1, EP 0 394 737 A 1, EP 0 590 484 A 1, EP 0 234 362 A 1, EP 0 234 361 A 1, EP 0 543 817 A 1, WO 95/14721, EP 0 521 928 A 1, EP 0 522 420 A 1, EP 0 522 419 A 1, EP 0 649 865 A 1, EP 0 536 712 A 1, EP 0 596 460 A 1, EP 0 596 461 A 1, EP 0 584 818 A 1, EP 0 669 356 A 1, EP 0 634 431 A 1, EP 0 678 536 A 1, EP 0 354 261 A 1, EP 0 424 705 A 1, WO 97/49745, WO 97/49747, EP 0 401 565 A 1 or EP 0 817 684, column 5, lines 31 to 45.

In the process of the invention it is possible in principle to use all conventional one-component (1K), two-component (2K) or multicomponent (3K, 4K) clearcoats, powder clearcoats, powder slurry clearcoats, or UV-curable clearcoats.

Thermally curable one-component (1K), two-component (2K) or multicomponent (3K, 4K) clearcoats are known from the European patent applications DE 42 04 518 A 1, EP 0 594 068 A 1, 0 594 071 A 1, 0 594 142 A 1, 0 604 992 A 1 or 0 596 460 A 1, from the international patent applications wO 94/10211, WO 94/10212, WO 94/10213, WO 94/22969 or WO 92/22615, or from the U.S. Pat. Nos. 5,474,811 A 1, 5,356,669 A 1 or 5,605,965 A 1.

One-component (1K) clearcoats are known to comprise hydroxyl-containing binders and crosslinking agents such as blocked polyisocyanates, tris(alkoxy-carbonylamino)triazines and/or amino resins. In a further variant they comprise as binders polymers having pendant carbamate and/or allophanate groups, and carbamate and/or allophanate-modified amino resins as crosslinking agents (cf. U.S. Pat. Nos. 5,474,811 A 1, 5,356,669 A 1 or 5,605,965 A 1, International Patent Applications WO 94/10211, WO 94/10212 or WO 94/10213, or European Patent Applications EP 0 594 068 A 1, 0 594 071 A 1 or 0 594 142 A 1).

Two-component (2K) or multicomponent (3K, 4K) clearcoats are known to comprise, as essential constituents, hydroxyl-containing binders and polyisocyanates as crosslinking agents, these constituents being stored separately until they are used.

Thermally curable powder clearcoats are known, for example, from German Patent Application DE 42 22 194 A 1 or from the product information bulletin from BASF Lacke+Farben AG, “Pulverlacke” [Powder coatings], 1990 or in the BASF Coatings AG brochure “Pulverlacke, Pulverlacke fur industrielle Anwendungen” [Powder coating materials, powder coatings for industrial application], January 2000.

Powder clearcoats are known to comprise, as essential constituents, binders containing epoxide groups, and polycarboxylic acids as crosslinking agents.

Thermally curable powder slurry clearcoats are known, for example, from U.S. Pat. No. 4,268,542 A 1 and from German Patent Applications DE 195 40 977 A 1, DE 195 18 392 A 1, DE 196 17 086 A 1, DE-A-196 13 547, DE 196 52 813 A 1 or DE-A-198 14 471 A 1 or described in German Patent Application DE 198 14 471.7, unpublished on the priority date of the present specification.

Powder slurry clearcoats are known to comprise powder clearcoats dispersed in an aqueous medium.

UV-curable clearcoats and powder clearcoats are disclosed, for example, in European Patent Applications EP 0 928 800 A 1, 0 636 669 A 1, 0 410 242 A 1, 0 783 534 A 1, 0 650 978 A 1, 0 650 979 A 1, 0 650 985 A 1, 0 540 884 A 1, 0 568 967 A 1, 0 054 505 A 1 or 0 002 866 A 1, German Patent Applications DE 197 09 467 A 1, 42 03 278 A 1, 33 16 593 A 1, 38 36 370 A 1, 24 36 186 A 1 or 20 03 579 B 1, International Patent Applications WO 97/46549 or 99/14254, or U.S. Pat. Nos. 5,824,373 A 1, 4,675,234 A 1, 4,634,602 A 1, 4,424,252 A 1, 4,208,313 A 1, 4,163,810 A 1, 4,129,488 A1, 4,064,161 A 1 or 3,974,303 A 1. Also known are clearcoats which can be crosslinked thermally and with actinic radiation (cf. European Patent Application EP 0 844 286 A 1).

Examples of suitable coating materials producing high scratch resistance sealers that are to be used for the process of the invention are described in German Patents DE 43 03 570 A 1, DE 34 07 087 A 1, DE 40 11 045 A 1, DE 40 25 215 A 1, DE 38 28 098 A 1, DE 40 20 316 A 1 or DE 41 22 743 A 1. Also suitable are organically modified ceramic materials which are sold under the brand name ORMOCER®.

This demonstrates that the process of the invention may be carried out with a very great diversity of different coating materials, which is a particular advantage.

In general, the coating materials are applied in a wet film thickness such that they cure to coats having the thicknesses necessary and advantageous for their functions. In the case of the powder coating this thickness is preferably from 30 to 300, more preferably from 35 to 250, with particular preference from 40 to 200, with very particular preference from 45 to 150, and in particular from 50 to 120 μm; in the case of the basecoat it is preferably from 5 to 50, more preferably from 5 to 40, with particular preference from 50 to 30, and in particular from 10 to 25 μm; and in the case of the clearcoat it is preferably from 10 to 100, more preferably from 15 to 80, with particular preference from 20 to 75, and in particular from 25 to 70 μm. High scratch resistance sealers generally have lower coat thicknesses of, for example, below 5.0 μm.

The process of the invention produces multicoat finishes which are superior to multicoat finishes produced in a manner not in accordance with the invention as regards surface smoothness, corrosion protection, substrate adhesion, intercoat adhesion, stonechip resistance, weathering stability, and chemical resistance. Surprisingly, they attain or exceed the level of the particularly weathering-resistant powder clearcoat finishes based on polyacrylate.

This is also the case for the multicoat finish comprising color and/or effect powder coating and clearcoat, which is economically and technically advantageous for the painting of utility vehicles. Since—as already mentioned above—a lack of weathering stability is manifested particularly rapidly and markedly with this coating system, this multicoat finish may be used as a model or test system for determining the weathering stability of multicoat finishes comprising color and/or effect powder coating, color and/or effect basecoat, and clearcoat. If the powder coating already has the required weathering stability in the test system, it will certainly have it in a system in which it is protected from the outside world by additional coating films.

The multicoat finishes of the invention therefore possess particularly high quality and a long service life even under extreme climatic conditions, thus making them especially attractive, economically and technically, to the user.

### EXAMPLES AND COMPARATIVE EXPERIMENTS

### Preparation Example 1

The Preparation, Application and Curing of the Powder Coating Materials

The powder coating materials used in carrying out Examples 1 to 4 and Comparative Experiments C1 and C2 were prepared by the following general procedure.

The powder coating material constituents listed in Table 1 were weighed out in accordance with the respective formulations. The batch size was 5.0 kg. The respective constituents were homogenized in a Henschel fluid mixer at 2,800 rpm for two minutes. After premixing using a funnel, the resulting mixtures were supplied to an extruder (BUSS PR 46). The extruder was operated in each case with a barrel temperature of 110° C., and the speed of rotation was chosen so that the barrel temperature stayed at 60° C. The emerging extrudate was cooled to 20° C. with a cooling roll and comminuted to chips using a breaker. The chips were precut and ground in a pin mill. The resulting powder was sieved off above 125 μm with a tumbler sieve. Application was carried out in each case with the aid of a manual laboratory gun with corona charging, of the Watner ESB type, in a film thickness of 60 to 90 μm onto steel panels coated with a conventional, cathodically deposited and baked CED. The resulting powder coating films were baked at 160-180° C. for 10-25 minutes.

Table 1 gives an overview of the formulations of the powder coating materials.

TABLE 1
The material composition of the powder
coating materials for use in accordance with
the invention (Examples 1 to 4) and of the
powder coating materials not for use in
accordance with the invention (Comparative
Experiments C1 and C2)
Composition (parts by weight):

| | | | Comparative |
|---|---|---|---|
| | | | Experiments |
| | Examples Nos.: | | Nos.: |
| | 1,2 | 3,4 | C1,C2 |
| Binders: | | | |
| Uralac ® P3480 ^{a)} | 55.4 | — | — |
| Uralac ® P3485 ^{a)} | | 54.87 | — |
| Alftalat ® AN 783 ^{b)} | — | — | 40 |
| Epikote ® 3003 | | | |
| FCA-10 ^{c)} | — | — | 7 |
| Araldit ® GT 6063 ^{e)} | — | — | 12 |
| Crosslinking agent: | | | |
| Araldit ® PT910 ^{d)} | 3.6 | 4.13 | — |
| Pigments: | | | |
| Carbon black, special black 4 | 0.1 | 0.1 | 0.1 |
| Titanium rutile 2310 ^{f)} | 11.3 | 11.3 | 11.3 |
| Blanc Fixe ® PLV.N ^{g)} | 26.64 | 26.64 | 26.64 |
| Bayferrox ® 130 BM/P ^{h)} | 0.99 | 0.99 | 0.99 |
| Irgalith-Blau ® PDS 6 ⁱ⁾ | 0.07 | 0.07 | 0.07 |
| Additives: | | | |
| Aerosil ® ^{j)} | 0.2 | 0.2 | 0.2 |
| Benzoin | 0.6 | 0.6 | 0.6 |
| Licowax ® R 21 ^{k)} | 0.1 | 0.1 | 0.1 |
| Tinuvin ® 144 ^{l)} | — | — | 0.5 |
| BYK ® 360 P ^{m)} | 1 | 1 | — |

^{a)} carboxyl-containing polyesters from DSM;
^{b)} carboxyl-containing polyester from Hoechst AG;
^{c)} epoxy resin from Deutsche Shell;
^{d)} mixture of terephthalic and isophthalic acid diglycidyl ester from Ciba;
^{e)} epoxy resin from Ciba;
^{f)} titanium dioxide pigment from Kronos International;
^{g)} barium sulfate pigment from Sachtleben;
^{h)} iron oxide pigment from Bayer AG;
ⁱ⁾ organic blue pigment from Ciba;
^{j)} pyrogenic silica from Clariant;
^{k)} lubricant from Clariant;
^{l)} light stabilizer from Ciba Specialty Chemicals;
^{m)} leveling agent from Byk Chemie.

### Preparation Example 2

The Production of Multicoat Finishes of the Powder Coating/Clearcoat Structural Type

The multicoat powder coating/clearcoat finishes described in Examples 2 to 4 and in the Comparative Experiment C2, described below, were prepared in accordance with the following general procedure.

The powder coatings prepared in accordance with Preparation Example 1 were overcoated using a conventional gravity-type gun with a commercial high-solids two-component clearcoat from BASF Coatings AG. After curing, the coat thickness was 35 to 45 μm. To aid comparability, the same clearcoat was used in each case for the examples and comparative experiments in question.

### Preparation Example 3

The Production of Multicoat Finishes of the Powder Coating/Basecoat/Clearcoat Structural Type

The multicoat powder coating/basecoat/clearcoat finishes described in Examples 1 to 3 and in the Comparative Experiment C1, described below, were prepared in accordance with the following general procedure.

The powder coatings prepared in accordance with Preparation Example 1 were coated using a conventional gravity-type gun with a commercial blue-black basecoat (Deep Amethyst FWO6-701C from BASF Coatings AG). To aid comparability, the same basecoat was used in each case for the examples and comparative experiments in question.

Following application, the basecoat film was flashed off and dried at 50° C. for 10 minutes. Thereafter it was overcoated in the manner described in Preparation Example 2 with the clearcoat used therein. Subsequently, the basecoat film and clearcoat-film were cured together at 145° C. for 30 minutes.

This gave basecoats having a thickness of from 12 to 15 μm and clearcoats having a thickness of from 35 to 45 μm.

### Examples 1 to 4 and Comparative Experiments C1 and C2

### Weathering Stability of the Multicoat Finishes Produced in Accordance With the Invention (Examples 1 to 4) and of the Multicoat Finishes Produced Conventionally (Comparative Experiments C1 and C2)

The weathering stability of the test panels produced in accordance with Preparation Examples 2 and 3 was determined in accordance with the CAM accelerated weathering test using Xenotest® apparatus from the company Atlas Material Testing Technology B.V., Netherlands. The weathering time was 2000 hours in each case.

In a first series, the adhesive strength of the multicoat finishes directly after weathering was tested by the cross-cut test and evaluated as follows: rating 0=no delamination; 1=slight delamination but still satisfactory; 2=partial delamination, not satisfactory; 3-4=extensive delamination, not satisfactory; 5=complete delamination. The results are given in Table 2 under Series 1.

In a second series, the adhesive strength of the multicoat finishes after weathering, 72-hour exposure to a constant humidity climate (SKK) and one hour's exposure in the rain was determined by the cross-cut test and rated as described above. The results are given in Table 2 under Series 2.

In a third series, the second series was tested again after 24-hour regeneration. The results are likewise given in Table 2, under Series 3.

Comparison of the results shows that the weathering stability of the multicoat powder coating/basecoat/clearcoat finish of Comparative Experiment C1 still corresponds approximately to that of the multicoat finishes of Examples 1 and 3. However, the weathering stability of the multicoat powder coating/clearcoat finish of Comparative Experiment C2 is definitely inferior to that of the multicoat finishes of Examples 2 and 4. In other words, the test in the powder coating/clearcoat system, which is harder for the powder coatings, unambiguously differentiates the powder coatings in accordance with their weathering stability.

TABLE 2
The weathering stability of the multicoat
finishes produced in accordance with the
invention (Examples 1 to 4) and not in
accordance with the invention (Comparative
Experiments C1 and C2)
Examples and Comparative Test results:

| Experiments Nos. | Series 1 | Series 2 | Series 3 |
|---|---|---|---|
| 1 | 0 | 0 | 0 |
| 2 | 1 | 1 | 1 |
| 3 | 0 | 0 | 0 |
| 4 | 0 | 5 | 5 |
| C1 | 0 | 0 | 0 |
| C2 | 5 | — | — |

### Preparation Example 4

The Preparation, Application and Curing of the Powder Coating Materials

The powder coating materials used to carry out Examples 5 to 12 and Comparative Experiments C3 to C5 were prepared by the general procedure specified in Preparation Example 1, applied and cured. Table 3 gives an overview of the material composition of the powder coating materials.

TABLE 3
The material composition of the powder coating materials for use in accordance with the
invention (Examples 5 to 12) and of the powder coating materials not for use in accordance
with the invention (Comparative Experiments C3 to C5)
Composition (parts by weight):

| | | | | | | | | Comparative | | |
|---|---|---|---|---|---|---|---|---|---|---|
| | | | | | | | | Experiment | | |
| Example Nos.: | | | | | | | | Nos.: | | |
| 5 | 6 | 7 | 8 | 9 | 10 | 11 | 12 | C3 | C4 | C5 |

Binders:

| | | | | | | | | | | | |
|---|---|---|---|---|---|---|---|---|---|---|---|
| Uralac ® 1480 ^{a)} | 52.8 | — | — | — | — | — | 52.8 | 50.9 | — | — | — |
| Uralac ® P 5000 ^{b)} | — | — | — | — | — | — | — | — | — | — | 54.6 |
| Alftalat ® AN 783 ^{c)} | — | — | — | — | — | — | — | — | 38.5 | — | — |
| Araldit ® GT 6063 ^{d)} | — | — | — | — | — | — | — | — | 20.2 | 14.7 | — |
| Uralac ® P 2450 ^{b)} | — | — | — | — | — | — | — | — | — | 44 | — |
| Uralac ® P 3475 ^{b)} | — | 54.6 | — | — | — | — | — | — | — | — | — |
| Uralac ® P 846 ^{b)} | — | — | 55.7 | 63.5 | 55.7 | 55.6 | — | — | — | — | — |
| Crosslinking agents: | | | | | | | | | | | |
| Vestagon ® BF 1540 ^{e)} | 5.9 | — | — | — | — | — | — | — | — | — | — |
| Vestagon ® BF 1300 ^{e)} | — | — | — | — | — | — | 5.9 | 5.7 | — | — | — |
| Araldit ® PT 910 ^{f)} | — | 4.1 | — | — | — | — | — | — | — | — | — |
| Primid ® XL 552 ^{g)} | — | — | 3 | 2.9 | 3 | 3 | — | — | — | — | — |
| Araldit ® PT 810 ^{h)} | — | — | — | — | — | — | — | — | — | — | 4.1 |
| Pigments: | | | | | | | | | | | |
| Titanium rutile 2310 ⁱ⁾ | 20 | 20 | 20 | 20 | 20 | 20 | 20 | 20 | 20 | 20 | 20 |
| Blanc Fixe ® PLV.N ^{j)} | 20 | 20 | 20 | 20 | 20 | 20 | 20 | 20 | 20 | 20 | 20 |
| Additives: | | | | | | | | | | | |
| Benzoin | 0.6 | 0.6 | 0.6 | 0.6 | 0.6 | 0.6 | 0.6 | 0.6 | 0.6 | 0.6 | 0.6 |
| BYK ® 360 P ^{k)} | 0.6 | 0.6 | 0.6 | 0.6 | 0.6 | 0.6 | 0.6 | 0.6 | 0.6 | 0.6 | 0.6 |
| Tinuvin ® 144 ^{l)} | 0.7 | — | — | 0.7 | — | — | — | 0.7 | — | — | — |
| Tinuvin ® 900 ^{l)} | 1.4 | — | — | 1.4 | — | — | — | 1.4 | — | — | — |
| Worlee Add ® 902 ^{m)} | — | — | — | — | — | 0.5 | — | — | — | — | — |

^{a)} hydroxyl-containing polyester from DSM;
^{b)} carboxyl-containing polyester from DSM;
^{c)} carboxyl-containing polyester from Hoechst;
^{d)} epoxy resin from Ciba;
^{e)} blocked, isophorone-based polyisocyanates containing uretdione groups, from Hüls;
^{f)} mixture of terephthalic and isophthalic acid diglycidyl ester from Ciba;
^{g)} tetrakis (beta-hydroxyethyl) adipamide from DSM;
^{h)} TGIC from Ciba;
ⁱ⁾ titanium dioxide pigment from Kronos International;
^{j)} barium sulfate pigment from Sachtleben;
^{k)} leveling agent from Byk Chemie;
^{l)} light stabilizer from Ciba Specialty Chemicals;
^{m)} leveling agent from Worlee.

### Preparation Example 5

The Production of Multicoat Finishes of the Powder Coating/Clearcoat Structural Type

Preparation Example 3 was repeated except that the powder coating materials used were those of Table 3 instead of those of Table 1 and that a different commercial conventional two-component clearcoat from BASF Coatings AG was used.

### Examples 5 to 12 and Comparative Experiments C3 to C5

### Weathering Stability of the Multicoat Finishes Produced in Accordance with the Invention (Examples 5 to 12) and of the Multicoat Finishes Produced Conventionally (Comparative Experiments C11 to C13)

The weathering stability of the test panels of Preparation Example 5 was determined immediately (series A) and following additional 240-hour SSK exposure (series B) after two years of outdoor weathering in Jacksonville, Fla., on the basis of the adhesive strength in the cross-hatch test in accordance with DIN ISO 2409: 1994-10.

The results can be found in Table 4.

They show that the multicoat finishes of Examples 5 to 12 produced in accordance with the invention have a weathering stability like that of the multicoat finish of Comparative Experiment C5 (formulation containing TGIC) and therefore offer an alternative to it. Moreover, the multicoat finishes of Examples 6 to 12 exhibit an extremely high level of adhesion between powder coating and clearcoat, so that delamination occurs only at the EDP/powder coating boundary. The corresponding cross-hatch values therefore relate to this delamination at said boundary.

In contrast, virtually complete, or complete, delamination between powder coating and clearcoat occurred as early as during weathering in the case of Comparative Experiment C3 and during the cross-hatch test in the case of Comparative Experiment C4.

TABLE 4
The weathering stability of the multicoat
finishes produced in accordance with the invention
(Examples 5 to 12) and of the multicoat finishes
produced not in accordance with the invention
(Comparative Experiments C3 to C5)
Cross-hatch testing (rating gt0-gt5):

| | | | | | | | | | Comp. | | |
|---|---|---|---|---|---|---|---|---|---|---|---|
| | Ex- | | | | | | | | Exper- | | |
| | ample | | | | | | | | iments | | |
| | Nos.: | | | | | | | | Nos.: | | |
| | 5 | 6 | 7 | 8 | 9 | 10 | 11 | 12 | C3 | C4 | C5 |
| A | gt0 | gt1 | gt1 | gt1 | gt2 | gt2 | gt2 | gt1-2 | X) | Y) | gt0 |
| B | gt0 | gt4 | gt5 | gt5 | gt5 | gt5 | gt4 | gt2 | X) | Y) | g0 |

X) The clearcoat flaked off as early as during Florida weathering;
Y) The clearcoat delaminated during the cross-hatch test.

What is claimed is:

1. A process for producing multicoat color and/or effect finishes on motor vehicle bodies or parts thereof (substrates), in which
(I) a pigmented powder coating material is applied to the substrates,
(II) the resulting powder coating film is partly or fully cured,
(III) the powder coating film or powder coating is overcoated with a solid-color topcoat, with a basecoat and a clearcoat, or with a clearcoat, after which
(IV) the resulting films are cured in each case individually or together with other films (wet-on-wet technique),
wherein said powder coating material
(A) comprises at least one carboxyl-containing polyester as binder and
(B) comprises at least one glycidyl ester of an aromatic or of a saturated or unsaturated cycloaliphatic dicarboxylic acid and/or at least one N,N,N′,N′-tetrakis(beta-hydroxyalkyl)alkane-dicarboxamide as crosslinking agent, or
(A) comprises at least one hydroxyl-containing polyester as binder and
(B) comprises at least one internally blocked uretdione of a diisocyanate as crosslinking agent.

2. The process as claimed in claim 1, wherein the aromatic dicarboxylic acid is terephthalic acid or isophthalic acid.

3. The process as claimed in claim 1, wherein the N,N,N′,N′-tetrakis(beta-hydroxyalkyl)alkanedicarboxamide used is N,N,N′,N′-tetrakis(beta-hydroxyethyl)-alkanedicarboxamide.

4. The process as claimed in claim 3, wherein the N,N,N′,N′-tetrakis(beta-hydroxyethyl)alkanedicarboxamide used is N,N,N′,N′-tetrakis(beta-hydroxyethyl)-adipamide.

5. The process as claimed in claim 1, wherein the two isocyanate groups of the diisocyanate differ in reactivity.

6. The process as claimed in claim 5, wherein one isocyanate group is attached to an alkyl radical and the other isocyanate group is attached to a cycloalkyl radical.

7. The process as claimed in claim 6, wherein the diisocyanate is isophorone diisocyanate.

8. The process as claimed in any of claims 1 to 7, wherein prior to process step (III) the pigmented powder coating film (II)
1. is either melted and cured, to give the powder coating; or
2. is melted and not cured at all, or is only part-cured, or
3. is not subjected to any further aftertreatment.

9. The process as claimed in claim 8, wherein the powder coating film (II) is melted and is cured together with an electrodeposition coat (EDC) present on the substrate and, if appropriate, with at least one further coat.

10. The process as claimed in claim 8 or 9, wherein in process step (IV)
1. the solid-color topcoat is cured alone or together with the powder coating film,
2. the basecoat film and the clearcoat film are cured individually in succession, the basecoat film is cured together with the clearcoat film, the basecoat film is cured together with the powder coating film and subsequently the clearcoat film is cured on its own, or the basecoat film is cured together with the powder coating film and the clearcoat film, and
3. the clearcoat film is cured on its own or together with the powder coating film.

11. The process as claimed in any of claims 1 to 10, wherein the clearcoat film or clearcoat is also overcoated with a high scratch resistance sealer, after which the sealer is cured together with the clearcoat film and, if appropriate, with at least one further, underlying coat, or on its own.
